(19) United States
(12) Patent Application Publication
(10) Pub. No.: US 2024/0082040 A1
(43) Pub. Date: Mar. 14, 2024

(54) FLEXIBLE COMPRESSION APPLYING WEARABLE

(71) Applicant: Julianne Quintana, Huntington Beach, CA (US)

(72) Inventor: Julianne Quintana, Huntington Beach, CA (US)

(21) Appl. No.: 17/943,948

(22) Filed: Sep. 13, 2022

(51) Int. Cl. A61F 5/058; A61F 5/01

(52) U.S. Cl. CPC A61F 5/05866; A61F 5/013

(57) ABSTRACT

A flexible compression applying wearable for a human hand is formed of crossing first and second elastic bands of fabric that are extendible over the back of the hand and have fold-over boundaries at front-to-back continuation edges of the hand. The fold-over boundaries can be positionally adjusted by shifting up and down along the front-to-back continuation edges of the hand so as to thereby apply respective compression forces to different bones and/or joints of the hand as desired. Fastenings of the wearable are preferably confined to interiors of multi-layered constructs so as to not irritate the user's skin.

### BACKGROUND

Many maladies can cause pain or discomfort in human limbs including, for example, inflammatory arthritis, rheumatoid arthritis (RA) and osteoarthritis (OA) in the hands. It is believed that application of external compression forces to the affected parts and/or immobilization of those parts can alleviate some of the pain or discomfort. A variety of compression-applying and/or joint immobilizing devices have been tried including splints, hard wraps and finger compressing gloves. For example, U.S. Pat. No. 5,005,824 issued Apr. 9, 1991 to Eichel proposes a device having a body of resilient spongy material positioned upon the palm portion of the hand at a position adjacent the root portions of the fingers, including that of the thumb. Eichel proposes that during sleep, the fingers will clench and compress the spongy material and that this compression will urge the muscles of the fingers to return to a relaxed state thus alleviating problems associated with sleep. The Eichel device has fixedly positioned bands terminating with Velcro™ patches (hook and pile patches) for securing the bands to one another and thus attaching the device to a substantially fixed position relative to the palm and wrist. Because of this construction, a dexterous second hand of the user is needed to attach the Eichel device to the arthritis-afflicted other hand. However, if both hands are severely afflicted with arthritis this may not be practical. The positions of the bands on Eichel's device cannot be changed. The in-palm spongy body consistently impedes dexterous use of the arthritis-afflicted hand, for example impeding free articulation of the thumb so it can make easy contact with any of the other fingers. It would be advantageous to have a compression applying wearable that can be attached to an afflicted hand without need for a dexterous second hand to secure the wearable and that can allow for dexterous use of the arthritis-afflicted hand to the extent possible in view of the ever-changing states of the affliction.

### BRIEF SUMMARY

A flexible compression applying wearable is provided having crossing elastic bands of fabric whose positions can be adjusted to variably apply compression to different parts of the hand as needed. More specifically, the bands can be independently shifted to selectively overlap or not overlap various joints and bones of the thumb (a.k.a. first finger), or of the pointer finger (a.k.a. second finger), of the wrist and of the pinky (a.k.a. fifth finger) so as to selectively apply varying degrees of compression or torque at the positions about those joints (e.g., above or below the radiocarpal joint, the carpometacarpal joints of the first, second and fifth fingers, and/or the metacarpophalangeal joints of the first, second and fifth fingers). Additionally, the bands can be independently shifted to selectively free some of those joints or bones for greater degree of movement in light of the current state of affliction and desire of the user to more freely use respective parts of the hand or not.

In one embodiment, the wearable is formed of overlapping plural layers of a light weight elastic textile (e.g., 410 g/m² per layer) wherein tacking glue and/or stitchings or other fastenings are contained internally between the overlapping layers so as to prevent such fastenings from irritating corresponding parts of the hand that are contacted by exterior parts of the overlapping layers. In one embodiment, aside from the crossing elastic bands (which define part of a Mobius strip), the wearable includes an attached thumb tube (with tacking glue and/or stitchings contained internally between its plural overlapping layers) where the thumb tube can be adjustably torqued in a desired direction so as to apply a directional torqueing force to the thumb. The thumb may be alternatively kept out of the thumb tube so as to be freed for delicate hand work that needs greater degrees of freedom.

This Brief Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

### BRIEF DESCRIPTION OF THE DRAWINGS

FIG. 1A is a top plan view of an X-ray showing the bones and skin outline of the right hand for purpose of explanation.

FIG. 1B is a top plan view of the X-ray of FIG. 1A and identifying some of the bones and joints for purpose of explanation.

FIG. 1C is a top plan view of the X-ray of FIG. 1A but this time showing an X/Y frame of reference and showing bands of a flexible compression applying wearable in accordance with the present disclosure crossing over the back of the hand such that the bands can be adjusted in the +Y and −Y directions to thus selectively apply varying degrees of compression at positions of some of the joints or to free up those joints for easier movement as desired.

FIG. 1D is a top plan view of the X-ray of FIG. 1A and similar to FIG. 1C but this time showing bands of the flexible compression applying wearable being shifted to cross over the back of the hand so as to selectively apply varying degrees of compression at positions of some others of the joints while freeing up the thumb for easier movement when desired.

FIG. 2A is a top plan view of one embodiment of the flexible compression applying wearable of FIG. 1C without a hand inserted therein and formed as a Mobius strip where a magnification shows elastic cross threads.

FIG. 2B is a top plan view of cut-outs for forming a first multi-layer flexible compression applying wearable in accordance with the present disclosure.

FIG. 2C shows the patterns of FIG. 2B folded into respective two-layer constructs.

FIG. 2D shows intermediate twisting of a first of the folded constructs for defining a Mobius strip and insertion of the second construct as a thumb-hole defining construct.

FIG. 3 is a flow chart of a method of forming a multi-layer flexible compression applying wearable in accordance with the present disclosure.

FIG. 4 is a flow chart of a method of using a flexible compression applying wearable in accordance with the present disclosure.

### DETAILED DESCRIPTION

Among various pain and/or discomfort maladies that can afflict the human hands, osteoarthritis (OA) is particularly onerous in that it can cause inflammation and pain in the skin of the hand as well as in the joints. OA is understood to be a long-term disorder that primarily affects joints but can also affect other parts of the body, including the skin, nerves and blood vessels. Typically it is a progressively debilitating disease. However its symptoms may come and go on a day to day basis as well as shifting concentration to different ones of the joints and/or portions of the skin. OA typically manifests with signs of inflammation where the affected joints become swollen, warm, painful, deformed and stiff, and the affected skin portions become sweaty and easily irritated. OA tends to be particularly debilitating early in the morning on waking or following prolonged periods of inactivity. Allowing the patient to engage in dexterous use of the hand or hands to the extent possible can help to thwart progression of the disease.

OA is often treated with use of joint-immobilizing splints and braces. Pain medications, as well as steroids, and NSAIDs may be used to help with symptoms. However, as noted above, allowing the patient to engage in dexterous use of the hand or hands to the extent possible can help to thwart progression of the disease as well as providing the patient with a better quality of life. So the joint-immobilizing splints and braces can be counterproductive in that they thwart dexterous use.

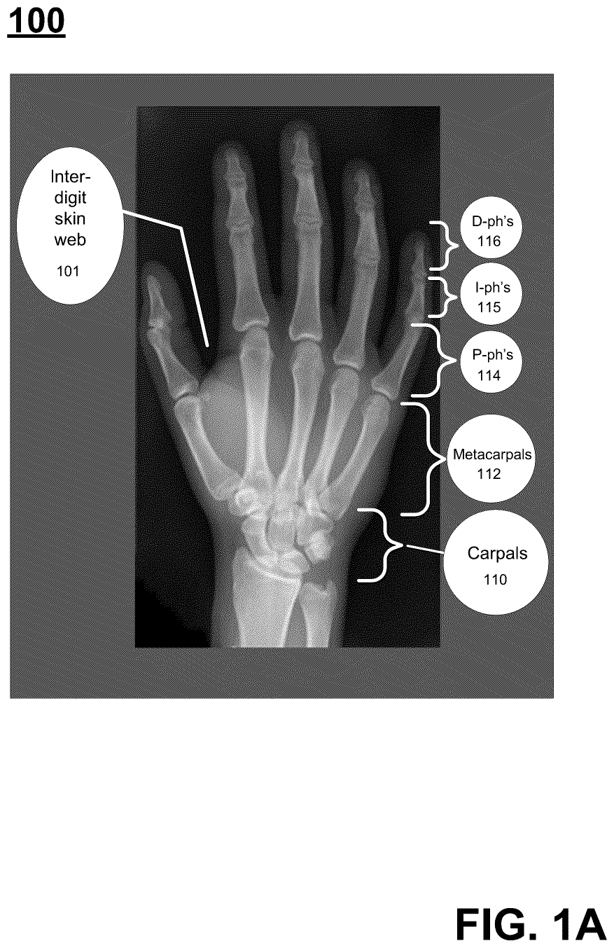

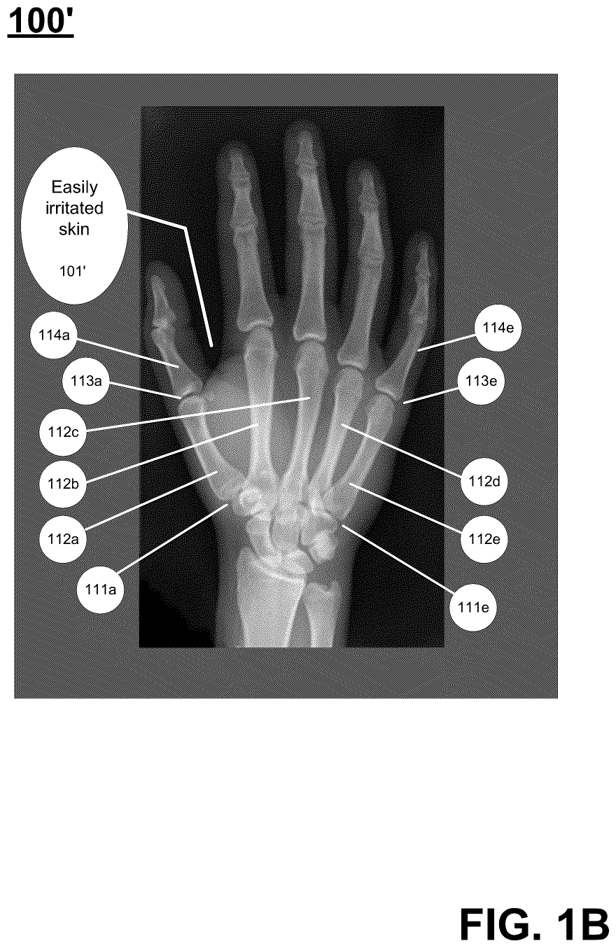

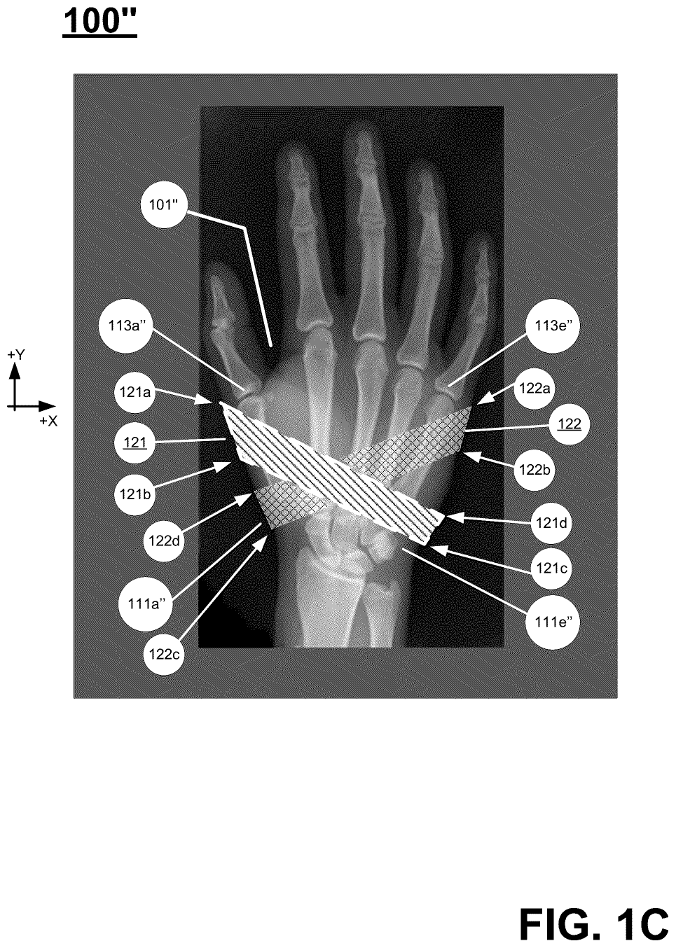

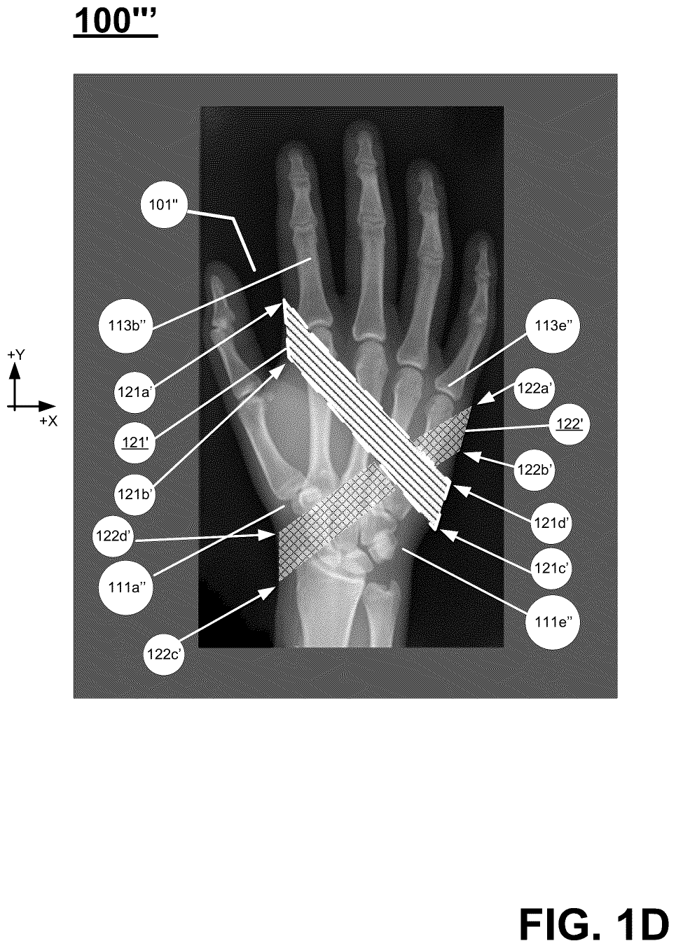

FIG. 1A is an annotated top plan view 100 of an X-ray showing the bones and the skin outline of the right hand (one that is not deformed by OA attack on the joints) for purpose of explanation. Starting at the bottom where the wrist articulates relative to the distal ends of the radius and ulna are a first set of bones 110 known as the carpals. Above them and overlapping the palm of the hand (latter not shown) are a second set of bones 112 known as the metacarpals. Some of the joints between the first and second sets of bones are identified in FIG. 1B as 111a (for providing for articulation of the first finger—the thumb—) and as 111e (for providing for articulation of the fifth finger—the pinky—).

Still referring to FIG. 1A, a third set of bones 114 known as the proximal phalanges (P-ph's) appear above the second sets of bones, with some of the joints in between being denoted as 113a and 113e (the metacarpophalangeals) in FIG. 1B. A fourth set of bones 115 known as the intermediate phalanges (I-ph's) appear above the third set. A fifth set of bones 116 known as the distal phalanges (D-ph's) appear above the fourth set. Any one or more of these joints may become inflamed and painful when arthritis strikes.

The human hand further includes webs of skin between the fingers, such as the interdigital skin web 101 illustrated between the first and second fingers (between the thumb and pointer finger).

Referring now more extensively to FIG. 1B, shown is a further annotated top plan view 100′ of the X-ray of FIG. 1A for identifying specific ones of the joints and establishing a system of reference. The first through fifth fingers are identified by the lower letter suffixes ‘a’ through ‘e’ such that the metacarpal of the thumb is denoted as 112a while that of the pointer finger is 112b; that of the middle finger is 112c; that of the ring finger is 112d and that of the pinky finger is 112e. Similarly, the P-ph of the thumb is denoted as 114a and the P-ph of the pinky is denoted as 114e. Respective joints of the thumb are denoted as 111a and 113a while respective joints of the pinky are denoted as 111e and 113e.

FIG. 1B additionally indicates that the interdigital skin webs; and in particular the web 101′ between the thumb and pointer finger can become easily irritated and painful when afflicted by OA. In accordance with one aspect of the present disclosure, it is desirable to avoid rubbing against the interdigital skin webs. Thus, compression applying wearables in accordance with the present disclosure preferably do not have components that will rub excessively against the skin webs when in use; and in particular that will avoid rubbing excessively against the web 101′ between the thumb and pointer finger.

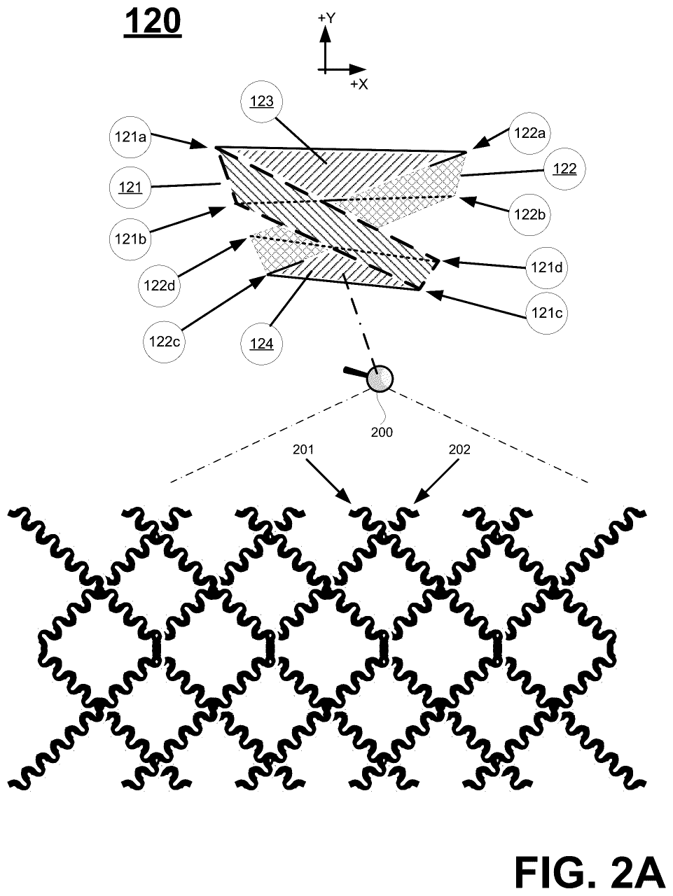

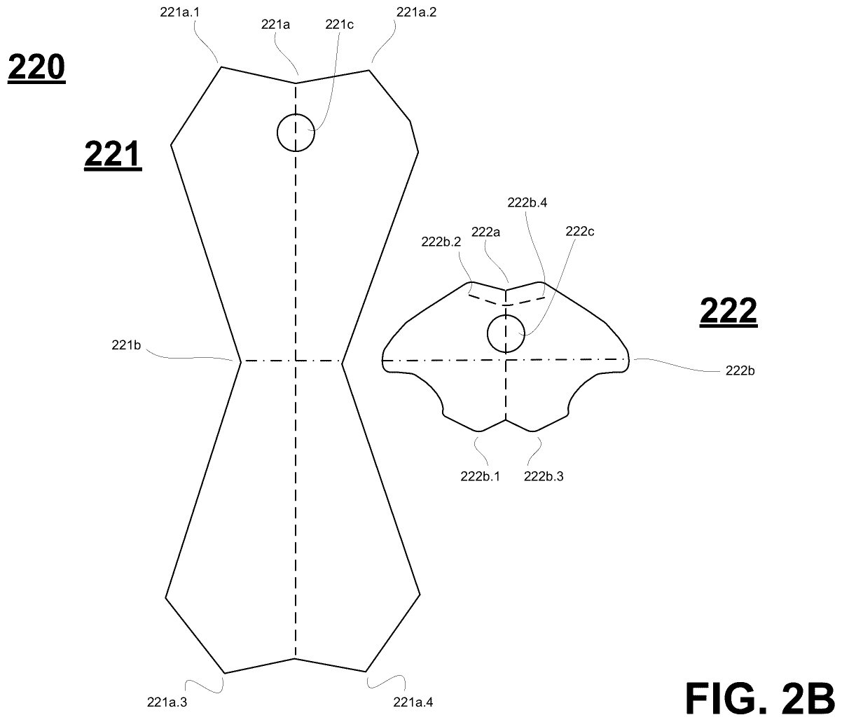

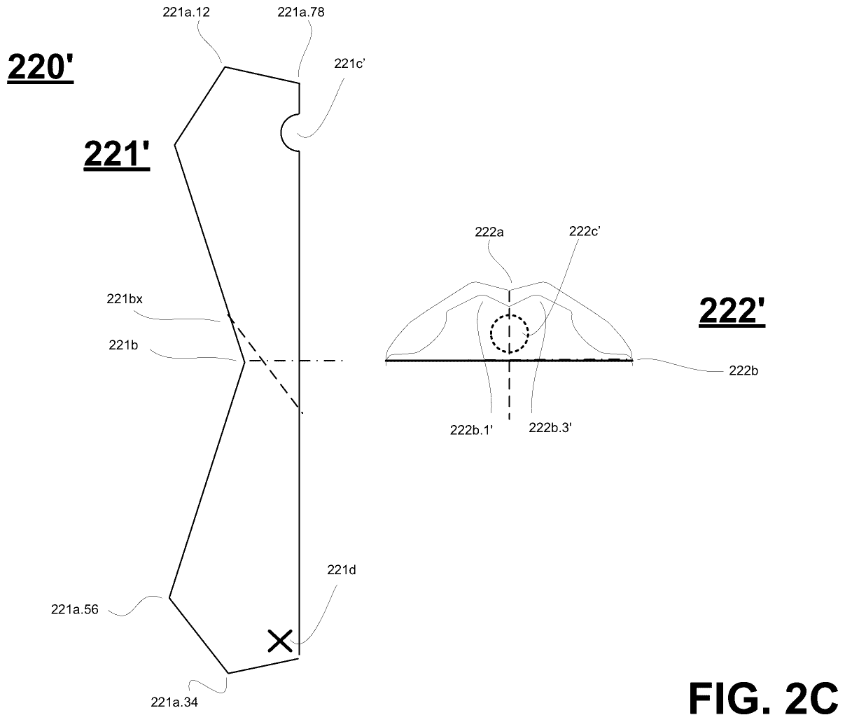

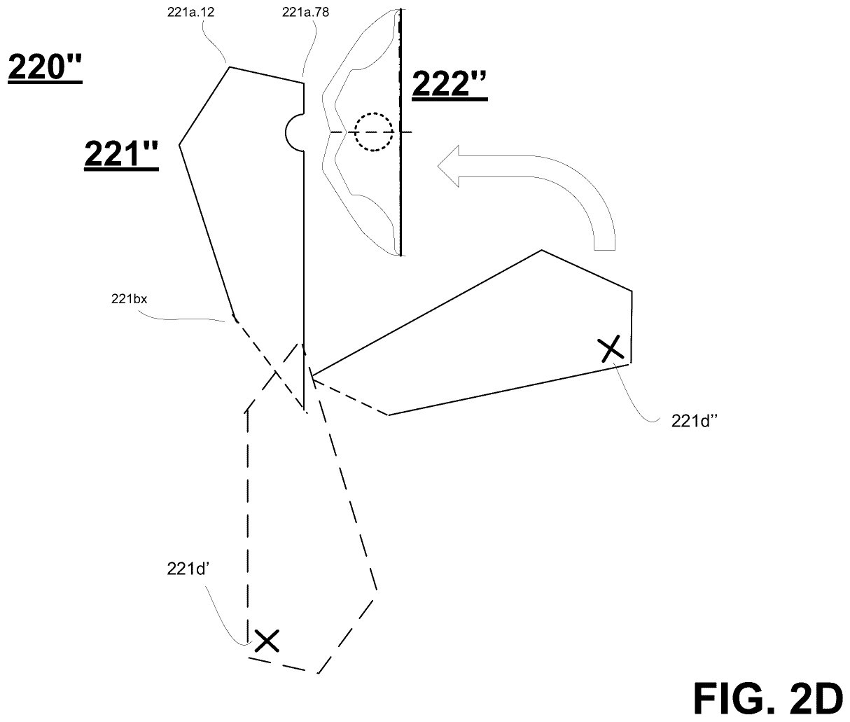

Referring to FIG. 1C, shown there is a top plan view of the X-ray of FIG. 1A but this time showing an X/Y frame of reference and showing first and second interconnected elastic bands 121 and 122 of a flexible compression applying wearable 120 (see also FIG. 2A) in accordance with the present disclosure. The shapes and sizes of the interconnected bands are not to scale. Some parts can be wider or narrower than what is shown. However, of importance, the illustrated fold-over boundaries; namely: line 121a-121b for the left side of first band 121, line 122c-122d for the left side of second band 122; line 122a-122b for the right side of second band 122 and line 121c-121d for the right side of first band 121 are angled to generally conform to the respectively contacted sides of the hand (typically an OA-deformed hand rather than the illustrated normal hand) so that instead of all of the fold-over boundaries of the bands defining straight lines that that perfectly at right angles to the major diagonal lines of those bands, they are inclined at hand-conforming other angles. These sloped angles of the fold-over boundaries may be created by cutting the bands in other than perfectly rectangular shape from a swatch of elastic fabric material. See briefly, FIG. 2B for an example of such non-rectangular shapes. See briefly, FIG. 2A for an understanding of how the above mentioned fold-over boundaries continue into the palm-side, counterpart and also-interconnected bands 123 and 124.

The first and second elastic bands 121 and 122 cross with one another and over the back of the hand such that the corners of their respective fold-over boundaries can be adjusted in the +Y and −Y directions to thus selectively apply varying degrees of compression at positions about some of the joints or to free up those joints and/or associated bones for easier movement as desired.

More specifically, the upper left corner 121a of the first band 121 may be shifted upwardly in the +Y direction from its position shown in FIG. 1C so as to instead be disposed above the base joint 113a″ of the thumb and to thus apply compressive force in the +X direction to the area above the base joint 113a″ of the thumb as desired. Of course this shift will somewhat impede free articulation of that joint depending on how far up in the +Y direction the upper left corner 121a is shifted. Similarly, the lower left corner 121b of the first band 121 may be shifted downwardly in the −Y direction from its position shown in FIG. 1C so as to be disposed further down along the thumb's metacarpal bone 112a (see FIG. 1B) and to thereby have less of a leverage advantage relative to the metacarpal-to-carpal joint 111a″ (also shown in FIG. 1B) so that the torque on that joint 111a″ is reduced.

By the same token, the upper left corner 122d of the second band 122 may be shifted upwardly in the +Y direction from its position shown in FIG. 1C so as to thereby have greater leverage advantage relative to the metacarpal-to-carpal joint 111a″. Or the lower left corner 122c of the second band 122 may be shifted downwardly in the −Y direction from its position shown in FIG. 1C so as to thereby apply pressure in the +X direction against the left side of the carpals 110. Any one or more of these adjustments may operate to reduce a current level of pain due to arthritis and also decrease or increase freedom of movement of the associated bones and/or joints.

Referring to the right side of the hand of the FIG. 1C, the upper right corner 122a of the second band 122 may be shifted upwardly in the +Y direction from its position shown in FIG. 1C so as to instead be disposed above the base joint 113e″ of the pinky and to thus apply compressive force in the −X direction to the area above the base joint 113e″ of the pinky if desired. Of course this shift will somewhat impede free articulation of that joint depending on how far up in the +Y direction the upper left corner 122a is shifted. Similarly, the lower left corner 122b of the second band 122 may be shifted downwardly in the −Y direction from its position shown in FIG. 1C so as to be disposed further down along the pinky's metacarpal bone 112e (see FIG. 1B) and to thereby have less of a leverage advantage relative to the metacarpal-to-carpal joint 111a (also shown in FIG. 1B) so that the torque on that joint 111e″ is reduced.

By the same token, the lower right corner 121c of the first band 121 may be shifted downwardly in the −Y direction from its position shown in FIG. 1C so as to thereby apply pressure in the −X direction against the right side of the carpals 110. The upper right side corner 121d of the first band 121 may be shifted upwardly in the +Y direction from its position shown in FIG. 1C so as to thereby have greater leverage advantage relative to the metacarpal-to-carpal joint 111e″ of the pinky side. Any one or more of these adjustments may operate to reduce a current level of pain due to arthritis and also decrease or increase freedom of movement of the associated bones and/or joints.

FIG. 1D is a top plan view of the X-ray of FIG. 1A and similar to FIG. 1C but this time showing bands of the flexible compression-applying wearable (now denoted as 121/122′) being shifted to cross over the back of the hand so as to selectively apply varying degrees of compression at positions of some others of the joints while freeing up the thumb for easier movement when desired. More specifically, fold-over boundary 121a′-121b′ of band 121′ is now elastically pressing against the second finger at joint 113b″ rather than against the thumb, thus freeing up the thumb for unopposed movement on days or other times when the afflicted person needs to have such freedom or is not as troubled by arthritis in the joints of the thumb. Note that the thumb's skin web 101′ is substantially not impinged on. Fold-over boundary 122c′-122d′ of band 122′ has been pulled lower down below the wrist to take up the slack resulting from freeing up the thumb. Thus the elastic compression against the second finger at joint 113b″ can be adjusted to be substantially the same as it was against the thumb before change of location of compression application from the thumb to the second finger. It is to be understood that the illustrated switch of location of compression application as between FIGS. 1C and 1D is merely an example. There could instead have been a removal or lessening of compression application to the fifth finger (pinky) by moving fold-over boundary 121c′-121d′ of band 121′ lower down below the wrist for example. Because the flexible compression applying wearable 121/122 is of a continuous construction (e.g., forming a three-dimensional (3D) FIG. 8—also known as a Mobius strip—when bands 121/122 as well as interconnected bands 123/124—see FIG. 2A—are appropriately slipped over the hand), the afflicted person does not have to battle with Velcro™ strips or other continuity-completing fasteners when making an adjustment. Compression change can often be realized simply by shifting one or more of the fold-over boundaries (e.g., 122c′-122d′) up or down along the respective side of the hand.

Referring to FIG. 2A, shown there is a top plan view of one embodiment of the flexible compression applying wearable 120 of FIG. 1C without a hand inserted therein. Like reference designations are used in FIG. 2A as in FIG. 1C to identify the fold-over boundaries; namely: line 121a-121b for the left side of first band 121, line 122c-122d for the left side of second band 122; line 122a-122b for the right side of second band 122 and line 121c-121d for the right side of first band 121. As can be seen, fold-over line 121a-121b defines a continuation into an under-palm, upper elastic band 123 whose opposed right end continues into fold-over line 122a-122b. Similarly, fold-over line 122c-122d defines a continuation into an under-palm, lower elastic band 124 whose opposed right end continues into fold-over line 121c-121d. The various bands, 121, 122, 123 and 124 do not have to be all of same widths or shapes. In one embodiment, the under-palm, lower elastic band 124 is substantially wider and associated fold-over lines 122c-122d and 121c-121d substantially longer than counterpart fold-over lines 121a-121b and 122a-121b. This allows the under-palm, lower elastic band 124 to more fully envelope the wrist. In one embodiment, a hole (not shown) is cut near the upper end of fold-over line 122c-122d so that the thumb may protrude from there. The flexible compression applying wearable 120 of FIG. 1C can be slipped onto an arthritis afflicted hand without having to open and close any continuation-completing fastener terminations (e.g., Velcro™ strips) since the bands are continuations, one into the other as shown in FIG. 1C. In one embodiment, rather than having an in-plane hole that merely surrounds the base of the thumb, a thumb-surrounding tube, made of the same elastic material, is attached to the structure (either as serial part of the plane of the Mobius strip or as outside the Mobius strip but still connected to it) so as to form a thumb-surrounding elastic structure that can be used to torque the thumb in a desired direction while no concentrating the torqueing force at one single line across the thumb.

In one embodiment, the material of one or more of the bands 121-124 (preferably of all of them, and also optionally of the thumb surrounding tube) is formed of a multi-directionally elastic soft fabric material. More specifically, as shown by magnification 200, elastic threads such as 201 and 202 are woven together to form an omni-directional spring system. Each of the threads such as 201 and 202 is twisted about its longitudinal axis to provide it with spring-like characteristics. The weave is relatively loose and porous so that sweat from the hand can easily evaporate into the ambient instead of being trapped by the material of the flexible compression applying wearable. The loose weave of the material allows it to be light weight. In one class of embodiments, the flexible compression applying wearable is formed of one or more layers of elastic textile having a mass density of no more than 1000 grams per square meter (g/m²) per layer. In one embodiment, the flexible compression applying wearable is formed of at least two overlapping layers of elastic textile each having a mass density of no more than 410 g/m². In one embodiment, the elastic textile layer(s) of the flexible compression applying wearable each have a fabric content of approximately 64% Nylon, 27% Polyester and 9% Spandex. The fabric may be provided as a Brazilian textured jacquard compression having an aesthetically pleasing coloration on one side (which becomes an exterior side) and a tacking-glue compatible opposed side (which becomes an interior side the fabric is folded into a multi-layer construct). In one embodiment, the material is made of cotton or a cotton-like jacquard weave. When parts of the fabric are fastened (e.g., stitched) together, a ¼ inch overlock joint seam and/or a tacking adhesive may be used. The cotton-like material of the textured jacquard compression is machine washable and able to wick-up moisture from the hand for evaporation into the ambient. The material may be decorated with various dye patterns for aesthetic purposes so that the compression applying wearable appears to be a fashion accessory. In one embodiment, fastenings such as sewn together layers or extensions of the elastic textile are tucked away inwardly, between the multilayers of the textile so as to not contact and thereby possibly irritate that user's skin. In one embodiment, the wearable comprises tubular constructs that can be flipped inside out due to their elastic nature. A majority of fastenings (e.g., stitchings) are formed before the outside-becomes-inside flipping occurs so that the fastenings are thereafter on the inside and away form the user's skin.

Referring to FIG. 2B, the flexible compression applying wearable 220 may be formed of plural segments like 221 and 222 cut out from a swatch of elastic fabric material and then folded, sewn or otherwise stitched together or tacked together with an appropriate textile glue or otherwise fastened. FIG. 2B is not to any specific scale because different sizes are used to accommodate hands of different sizes. In one embodiment, the wearables are formed as three relative sizes denoted as small, medium and large. The plural segments 221 and 222 of FIG. 2B are to scale relative to one another.

A method of forming a dual layer construct from the first cutout 221 includes folding along the longitudinal center axis 221a so as to bring corner 221a.1 into overlapping union with corner 221a.2 and so as to also bring corner 221a.4 into overlapping union with corner 221a.4. Slimmed-in waist line 221b is approximately where the twist of forming the Mobius strip is applied. Through-hole 221c may be used for alignment purpose. It also allows washing machine liquid to access the interior sides of the folded-together layers.

A method of forming a dual layer construct from the second cutout 222 includes folding along the horizontal axis 222b so as to bring corner 222b.1 into overlapping union with the left end 222b.2 of the illustrated hypothetical curve and so as to also bring corner 222b.3 into overlapping union with the right end 222b.4 of the illustrated hypothetical curve. Real curve 22a is then folded over and tacked to corners 222b.1 and 222b.3 so as to form a tube like construct, After most fastening are formed, the elastic material of the tube is then inverted inside-out (or more literally, such that the outside becomes an inside) so that fastenings such as the tacking glue is confined between the interior layers of the inverted tube. Hole 222c may be used for alignment purpose. It also allows washing machine liquid to access the interior sides of the folded-together layers.

FIG. 2C shows the folded constructs 221′ and 222′. In a subsequent step, after a Mobius-forming twist is made at 221bx, corners 221a.34 and 221a.56 will be joined (directly or indirectly) with corners 221a.12 and 221a.78. The “X” mark 221d is for alignment purposes and may be visible or hidden

FIG. 2D shows intermediate formations as dashed lines. The “X” mark (now denoted as 221d′) is on the other side after the Mobius-defining twist 221bx is applied. The “X” mark (now denoted as 221d″) rotates to its original side as the Mobius strip is closed together. In one embodiment, the thumb hole forming construct 222″ is serially included in the closed structure that defines the Mobius strip. In an alternate embodiment, it is attached as an out-of-plane construct sticking out orthogonally from the plane of the Mobius strip. Although FIG. 2B discloses the use of just two cutout patterns, it is within the contemplation of the present disclosure to connect together more than just two such patterns, each folded into a multi-layer construct. In one embodiment, a third pattern originally shaped a bow tie shape, is folded and formed into a tube. The tube is spirally folded to form a 3D cushion construct and the cushion is attached to a portion of the Mobius strip that merits extra cushioning for comforts sake (e.g., a wrist portion that may lean against a desk or other surface while hand work is performed). In one embodiment, a thumb surrounding tube is formed and attached orthogonally and serially in the plane of the Mobius strip or orthogonally but parallel to the plane of the Mobius strip. Attachment strips are preferably tucked into the insides of to-be-joined inverted tubes (whose outsides have become their insides) and are connected to the joined together inverted tubes by glue injected not the insides and dried in-situ there. Thus, none or essentially none of the fastenings impinge on the user's skin.

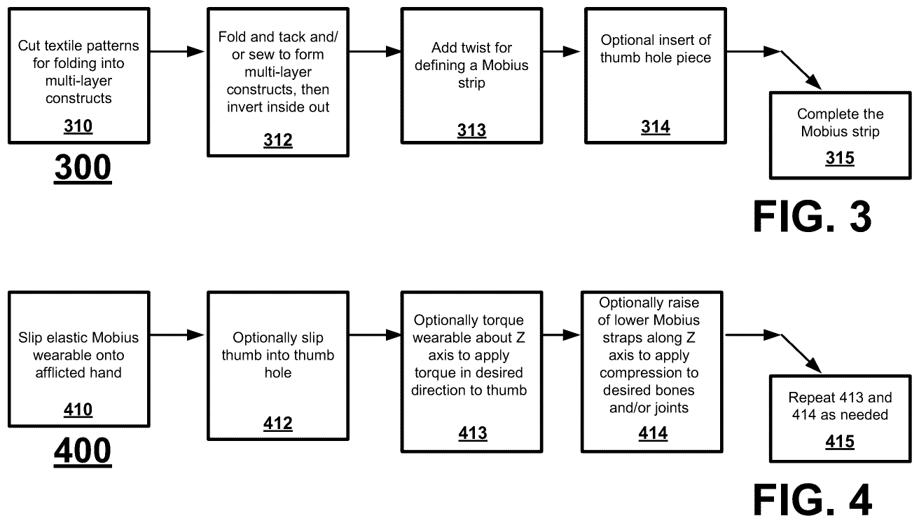

FIG. 3 illustrates a flow chart 300 of a method of forming a multi-layer flexible compression applying wearable in accordance with the present disclosure. Single layer textile sheets of elastic material are cut to form foldable patterns in step 310. Parts of the folded patterns can then be stitched or otherwise interconnected to form multi-layer constructs (e.g., tubular structures) in step 312. The elastic constructs are inverted inside out to confine the stitchings (and/or adhesive tackings or other fastenings) to the interiors between the layers so as to prevent the fabric connecting portions from irritating the wearer's skin. In step 313, a twist is added to one of the inverted constructs for the purpose of forming an elastic Mobius strip. Step 314 is optional for adding a thumb hole (and/or other finger restraining) construct either serially into the loop of the Mobius strip or as sticking out like an arm from the loop. In step 315 the loop is closed and the wearable is ready for use.

FIG. 4 illustrates a flow chart 400 of a method of using a flexible compression applying wearable in accordance with the present disclosure. In step 410, the loop of the already formed Mobius strip is slipped onto the afflicted hand without need for closing any attaching devices such as Velcro straps, zippers, lacings or the like. Step 412 is optional for inserting a thumb and/or other finger into a corresponding finger restraining construct. Step 413 is optional for torqueing the wearable about the longitudinal axis of the arm (the Z axis) to thereby apply elastic torque in a desired direction to the optionally inserted finger(s). Step 414 is optional for raising and/or lowering desired parts of the Mobius strip along the Z axis for thereby applying more or less compressive forces to respective bones and/or joints of the hand in accordance with what the wearer deems to be the most currently comforting configuration. In step 415, the desires of the wearer may change and then steps 413 and/or 414 are repeated to discover a currently more comforting configuration for the wearer.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

What is claimed is:

1. A flexible compression applying wearable for a human hand comprising:
crossing first and second elastic bands of fabric extendible over a back of the hand and having fold-over boundaries at front-to-back continuation edges of the hand that can be positionally adjusted by shifting up and down along the front-to-back continuation edges of the hand so as to thereby apply respective compression and/or torqueing forces to different bones and/or joints of the hand.

2. The flexible compression applying wearable of claim 1 wherein:
the crossing first and second elastic bands define parts of a Mobius strip.

3. The flexible compression applying wearable of claim 2 wherein:
the Mobius strip defines a permanently closed loop.

4. The flexible compression applying wearable of claim 3 wherein:
the Mobius strip is defined by overlapping layers of elastic fabric.

5. The flexible compression applying wearable of claim 4 wherein:
each of the overlapping layers of elastic fabric has density of no more than 1000 grams per square meter (g/m²).

6. The flexible compression applying wearable of claim 5 wherein:
each of the overlapping layers of elastic fabric has density of no more than 410 g/m².

7. The flexible compression applying wearable of claim 5 wherein:
each of the overlapping layers of elastic fabric is formed as Brazilian textured jacquard compression.

8. The flexible compression applying wearable of claim 5 wherein:
each of the overlapping layers of elastic fabric has a fabric content of approximately 64% Nylon, 27% Polyester and 9% Spandex.

9. The flexible compression applying wearable of claim 5 wherein:
at least two of the overlapping layers of elastic fabric are formed from a folded single layer of the elastic fabric.

10. The flexible compression applying wearable of claim 4 wherein:
one or more stitchings and/or other fabric to fabric fastenings are contained internally between two of the overlapping layers of elastic fabric.

11. A method of applying a flexible compression applying wearable to a human hand wherein the wearable comprises:
first and second elastic bands of fabric extendible disposed to cross over a back of the hand, the bands having fold-over boundaries at front-to-back continuation edges of the hand that can be positionally adjusted by shifting up and down along the front-to-back continuation edges of the hand so as to thereby apply respective compression forces to different bones and/or joints of the hand; and
wherein the Mobius strip defines a permanently closed loop;
the method compromising:
slipping the hand into the permanently closed loop such that the first and second elastic bands contact the back of the hand and such that another part of the permanently closed loop contacts a front of the hand.

12. The method of claim 11 and further comprising:
changing respective positions of one or both of the bands relative to the hand to thereby apply respectively changed compression forces to different bones and/or joints of the hand.

13. The method of claim 11 and further comprising:
slipping a finger of the hand into a finger-accepting hole or tube provided as part of the wearable; and
torqueing the finger-accepting hole or tube so as to thereby apply a desired elastic torqueing force to the finger.

14. The method of claim 13 wherein the finger is a thumb.

15. The method of claim 13 wherein the finger-accepting hole or tube is defined by a multi-layer fabric construct not having any fabric fastening parts on portions thereof contacting the slipped-in finger.

16. A method of making a flexible compression applying wearable to be worn on a human hand, the method comprising:
cutting from a sheet of elastic fabric one or more foldable patterns;
folding the foldable patterns;
fastening together two or more loose portions of each of the folded patterns to thereby define invertible constructs;
inverting the invertible constructs inside out so that the fastenings of the fastened together loose portions are disposed in interiors of the inverted constructs;
twisting one of the inverted constructs so as to define part of a Mobius strip; and
forming a permanently closed loop with one or more of the inverted constructs to thereby define the Mobius strip.

17. The method of claim 16 wherein:
each of the inverted constructs is defined overlapping layers of elastic fabric; and
each of the overlapping layers of elastic fabric has density of no more than 1000 grams per square meter (g/m²).

18. The method of claim 17 wherein:
the elastic fabric is formed as Brazilian textured jacquard compression.

19. The method of claim 17 wherein:
the elastic fabric has a fabric content of approximately 64% Nylon, 27% Polyester and 9% Spandex.
